(19) United States
(12) Patent Application Publication
(10) Pub. No.: US 2004/0202686 A1
(43) Pub. Date: Oct. 14, 2004

(54) Methods for creating customized hair care products

(76) Inventor: Mark Stephen Welch, West Chester, OH (US)

Correspondence Address: SCHWEGMAN, LUNDBERG, WOESSNER & KLUTH, P.A., P.O. BOX 2938, MINNEAPOLIS, MN, 55402, US

(21) Appl. No.: 10/410,052

(22) Filed: Apr. 9, 2003

(51) Int. Cl. G06F 17/60; A61K 7/06

(52) U.S. Cl. 424/401; 705/2

(57) ABSTRACT

Method and systems are provided for creating customized hair care products. A hair type is analyzed to determine scores associated with a plurality of factors. A base hair care product is acquired and a mixture of botanicals or other hair modifiers is generated based on the scores. The base hair care product and the mixture of botanicals are blended to produce the customized hair care product. In some embodiments, other mixtures are added in order to provide customize colors and fragrances to the customized hair care product. Further, the customized hair care product can be shampoo and/or conditioner.

### FIELD OF THE INVENTION

The present invention is related to customizing hair care products, and more specifically related to methods for creating and delivering customized hair care products.

### BACKGROUND OF THE INVENTION

Providing quality/healthy hair care products are rapidly growing industries in the world. Individuals are increasingly demanding individualized care and attention in order to address concerns they have over the health and appearance of their hair. A variety of products exist in the marketplace to address and meet some of these demands, such as shampoos, conditioners, gels, hairspray, coloring products, hair growth products, and others.

An individual's hair is a personal characteristic associated with an individual. In general, no two individuals will have the exact same characteristics or desires with respect to their hair health needs and their desired hair appearances. Yet, conventionally manufacturers and service providers have attempted to create a limited number of hair care products that are designed to address the most common health needs and desired appearances for a majority of individuals. Thus, even though individuals have plethora products to choose from, there currently is not any hair care product that is specifically customized and designed for an individual's hair type and an individual's specific desired appearances.

Furthermore, many individuals are very particular about the health and appearance of their hair. For many, having their hair professionally tended to and treated is as necessary and important as taking a shower or brushing teeth. Individuals want individual attention, analysis, and assistance with their hair styles and appearance. But, this same attention is not presently available to individuals in order to develop customized hair care products that are specifically designed and created based on an individual's hair type and an individual's hair appearance desires.

Accordingly, there is a need for improved techniques to create and deliver customized hair care products. The techniques should be tailored to the individual needs of one's hair type and to the individual desires of one's preferred hair appearance.

### SUMMARY OF THE INVENTION

Briefly and in general terms, methods and systems are provided for creating customized hair care products. The customized hair care products are created based on specific scores associated with factors, where the scores are acquired through analysis of a specific hair type. The scores are used to generate a mixture of botanicals (or other hair care modifiers) that is added to a base hair care product in order to generate the customized hair care product. In some embodiments, the customized hair care product can be shampoo and/or conditioner.

More specifically, and in one embodiment, a method to produce a customized hair care product is presented. A base hair care product is received. Furthermore, a score for a hair type is received, wherein the score is associated with a plurality of factors. The score is used to acquire a mixture of botanicals. Moreover, the mixture is added to the base to produce the customized hair product.

In still another embodiment, another method to produce a customized hair care product is described. A hair type is analyzed based on a plurality of factors. A prescription is generated for a customized hair care product based on scores assigned to the plurality of factors. A base hair care product is blended with a mixture of botanicals, which are determined from the prescription. Further, a resulting mixture is used from the blending to produce the customized hair care product.

In yet another embodiment, a customized hair care product is provided. The customized hair care product includes a base hair care product and a mixture of botanicals. The mixture of botanicals is determined by a prescription associated with a specific hair type. Furthermore, a percentage of the base hair care product and a percentage of the mixture are identified in the prescription in order to produce the customized hair care product.

Still other aspects of the present invention will become apparent to those of ordinary skill in the art from the following description of various embodiments. As will be realized the invention is capable of other embodiments, all without departing from the present invention. Accordingly, the drawings and descriptions are illustrative in nature and not intended to be restrictive.

### BRIEF DESCRIPTION OF THE DRAWINGS

FIG. 1 is a flowchart of a method for creating a customized hair care product, according to one embodiment of the present invention.

FIG. 2 is a flowchart of another method for creating a customized hair care product, according to one embodiment of the present invention.

FIG. 3 is a diagram of a customized hair care product, according to one embodiment of the present invention.

### DETAILED DESCRIPTION OF THE INVENTION

In the following description and the drawings illustrate specific embodiments of the invention sufficiently to enable those of ordinary skill in the art to practice it. Other embodiments may incorporate structural, logical, electrical, process, and other changes. Examples merely typify possible variations. Individual components and functions are optional unless explicitly required, and the sequence of operations may vary. Portions and features of some embodiments may be included in or substituted for those of others. The scope of the invention encompasses the full ambit of the claims and all available equivalents. The following description is, therefore, not to be taken in a limited sense, and the scope of the present invention is defined by the appended claims.

In various embodiments of the present invention, hair care base products are commercially available concentrations of basic or base shampoos and/or conditioners. These base hair care products are readily available and known to one of ordinary skill in the art. Furthermore, botanicals include oils, cholesterols, minerals, vitamins, herbal extracts, organic chemicals, selenium, medicated chemicals, pearlizing agents and the like. Fragrances and colorants include chemicals readily known and available to one of ordinary skill in the art.

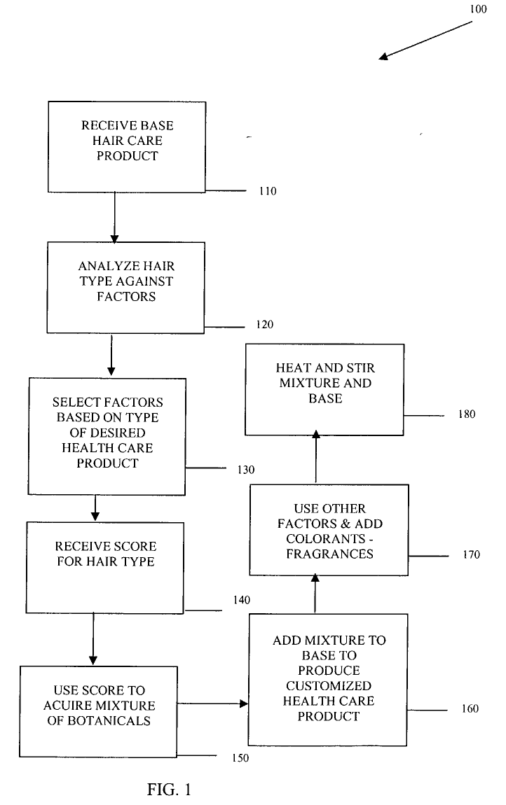

FIG. 1 illustrates a flowchart of one method 100 for creating a customized hair care product, according to one embodiment of the present invention. The method 100 can be automated and implemented in a computer accessible medium using one or more computing devices. Moreover, in some embodiments, the method 100 can be practiced in a combination of computer readable media with or without manual intervention.

At 110 a base hair care product is acquired, this can be acquired manually or in an automated fashion. The base hair care product is a concentrated shampoo and/or conditioner, depending upon what desired customized hair care product is being created. In fact in some embodiments, the base hair care product is a combination of shampoo and conditioner. The base can be available in a kiosk device for automatic acquisition in creating the desired customized hair care products. In other embodiments, the base is acquired from a hair care specialist or a technician that is creating the customized hair care product following the teachings of the present disclosure.

Initially, at 120, an individual's hair type is analyzed. This analysis can be done by a hair care specialist (e.g., licensed cosmetologist with or without special training and/or certification), to perform the analysis and make recommendations as to the formulation of the custom hair care product. Alternatively, the analysis can be driven by a series of automated and electronic questions posed to the individual regarding his/her hair type with or without the guidance of a licensed cosmetologist. The automated electronic questions can be posed to the individual via an interface associated with a kiosk, or the electronic questions can be accessed via a website from any computing device, within a salon, or at any location desired by the individual. In some embodiments, the analysis is partially performed automatically by one or more sensors that evaluate a strand of hair to detect various characteristics of the hair type (e.g., light, texture, dryness, porosity, elasticity, condition, shine, body, and the like).

The analysis at 120 is performed against a number of factors. The factors are selected based on the type (e.g., shampoo and/or conditioner) of desired customized hair care product that is being created. Thus, in one embodiment, when the desired customized hair care product is a shampoo then an individual's hair type is analyzed based on seven factors: a conditioning factor, a clarifying factor, a medicated factor, a body building factor, a relaxant factor, a shine factor, and a color preserving factor. In other embodiments, the seven factors can be a health/porosity factor, a texture factor, scalp dryness or oiliness factor, a body factor, an elasticity factor, a density factor, and a wave factor. In an embodiment where the desired customized hair care product is a conditioner, the hair type is analyzed based on four factors: frizz control, color preserving, body building, and deep conditioning.

One of ordinary skill in the art appreciates that the exact number of factors and combination of factors can be altered without departing from embodiments of the present invention. The factors are used to provide an objective analysis of an individual's hair type in order to acquire a proper mixture of botanicals to mix with the base hair care product. The analysis results in a score or prescription for the correct botanical mixture.

Thus, upon conclusion of the analysis, the hair type receives a score/prescription at 140. This score or prescription can be a single composite score or a series of scores for each evaluated factor vis-à-vis the hair type under evaluation. For example, each available factor can be associated with a numeric scale of integers from 0 to 10, such that a hair type under analysis receives an integer number score between 0 and 10 for each available factor.

By way of example only, consider a desired customized hair care product that is a shampoo, 7 factors can be defined on a scale of 0 to 10 as follows:

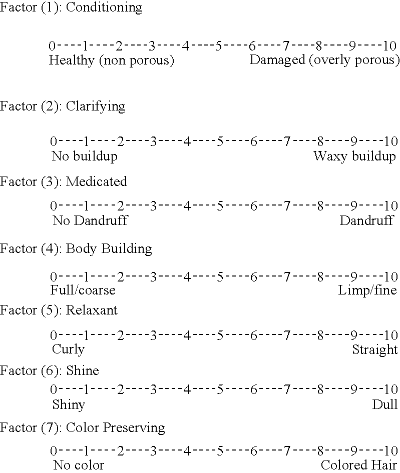

The scale can be more granular than from 0 to 10 or can include a larger range of numeric values (e.g., greater than 10). Once a hair type is analyzed and with respect to the appropriate factors then a final score, set of scores, or prescription for the hair type can be generated at 140. The score will map to appropriate concentrations and selections of botanicals at 150 designed to correct unhealthy conditions that were identified by the analysis of the hair type against the factors. It is readily known to one of ordinary skill in the art, what types of botanicals will assist in alleviating or mitigating certain unhealthy hair characteristics.

At 160, the identified botanical mixture acquired from the resulting score or prescription is added to the base hair care product and blended to produce a desired customized hair care product. In some embodiments, this blending or mixing can occur by first diluting or adding water to the concentrated base, adding brine, and then adding a number of the botanicals and/or other modifiers individually or as a mixture to the diluted base.

In some embodiments, at 170, other cosmetic and individually desired factors can be used to also add colors, fragrances, and/or pearlescence that are desired by an individual to further customize not only a healthy mixture for the customized hair care product, but an appealing product to the senses of the individual.

Furthermore, at 180, the resulting mixture can be heated to a desired or designed temperature and stirred appropriately to acquire a desired consistency and texture for the created customized hair care product. The process of analyzing and creating the customized hair care product can be automated within a kiosk, or partially automated where various aspects of analysis or creation occur via computing devices. In other embodiments, a variety of external or internal chemical agents can be used to mix the created customized hair care product to a desired thickness, consistency, and the like. All such external and internal mixing agents or techniques are intended to fall within the broad scope of the present invention.

For example, an individual can use a web camera to submit a picture of their hair to a hair care specialist (e.g., licensed cosmetologist, and the like) over a web connection. The individual can also answer a number of questions directed to the factors being used to determine the appropriate botanical mixture. With this information a prescription can be provided electronically to the individual, the prescription can then be filled by technicians that manually create the customized hair care product based on the directives of the prescription.

In still other embodiments, an individual visits a salon where their hair is analyzed against factors by a hair care specialist (e.g., licensed cosmetologist, and the like). The individual can also add a number of attributes or factors associated with desired smell and sight characteristics for the customized hair care product. The hair care specialist then writes a prescription (provides a composite score or series of scores for the hair type analysis vis-à-vis the factors). The individual takes the prescription to a technician within the salon or elsewhere and a base is diluted with water, thickened with brine, and a proper mixture of botanicals, colorants, pearlescence, fragrances, and/or other modifiers are blended into the diluted base. The resulting mixture is the customized hair care product that is directed to the specific hair type of the individual.

In yet more embodiments, a kiosk is designed to receive payment, interface with an individual, create a customized hair care product, and dispense the customized hair care product. Thus, for example, an individual selects a type of desired hair care product (e.g., shampoo, conditioner, a combination of shampoo and conditioner), and provides either a hair sample, or proceeds to take a questionnaire designed to extract characteristics from the individual in order to generate a score for factors. In other embodiments, a licensed and trained cosmetologist, who is certified to perform the hair analysis and make recommendations as to the proper formulation for the hair care product, assists the individual with the automated questionnaire either directly or virtually via a network connection. The score is then used to acquire a proper mixture of botanicals and/or other modifiers that are blended with a base. Colorants and Fragrances can also be blended. Next, the kiosk dispenses the customized hair care product in a container, which can either be acquired from the individual and subsequently used or provided at the kiosk with standard labeling and notifications.

It is now apparent, how a customized and specially designed hair care product can be created and distributed to individuals for use. This customized hair care product is not mass produced, but rather geared to an individual's specific hair needs and sensory desires. This is a significant improvement over what has been done in the past, since now individuals receive individualized and specific analysis of their hair type, which is used to create a specialized hair care product. Many individuals desire and seek individual and specialized hair care treatments, and thus teachings of the present invention will enhance and fulfill those desires.

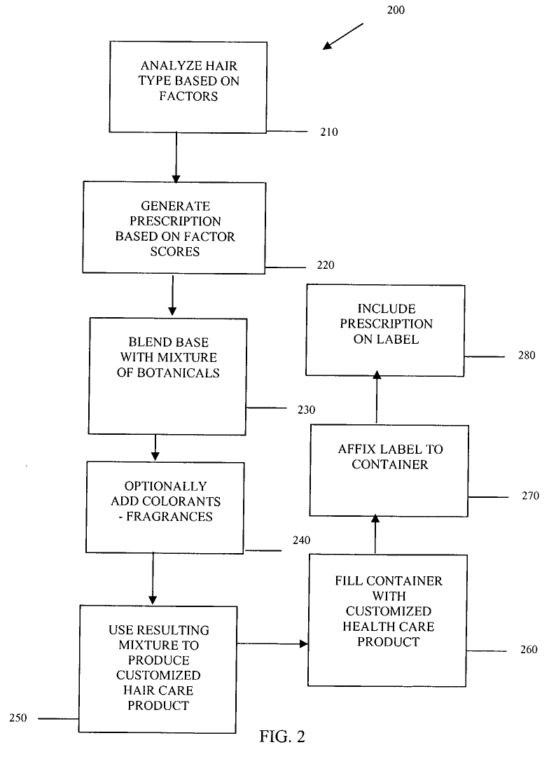

FIG. 2 illustrates a flowchart of another method 200 for creating a customized hair care product, according to one embodiment of the present invention. Method 200 can be fully implemented within one or more computer accessible media and/or partially implemented with one or more computer accessible media, which some processing occurring with manual intervention.

At 210, a hair type from an individual is analyzed with respect to a number of factors. The factors can vary depending upon the desired hair care product. Thus, one set of factors may be different when an individual desires a shampoo from another set of factors that are used when an individual desires a conditioner. Factors can be any objective measure that is used by one of ordinary skill in the art to evaluate the condition and characteristic of hair types. The application of the analysis to the objective factors can be objective such as when it is done in an automated fashion or partially subjective such as when done by a hair care specialist (e.g., licensed and trained cosmetologist, and the like).

Once the individual's hair type is analyzed vis-à-vis the factors, a prescription for the desired customized hair care product is generated at 120. The prescription represents the results of the analysis; it can be represented and presented in any standard form that can be consistently translated. Thus, the prescription can be a single score, a series of scores, or other formal representations developed to consistently and uniquely represent the analysis results.

The prescription can be produced by a hair care specialist interfacing with a software application, which is designed to produce the prescription based on values provided by the hair care specialist. In other embodiments, the prescription is entirely automatically generated, such as by a kiosk or individual interactions with an automated website, and the like. In still further embodiments, the prescription is generated by a hair care specialist using a number of guides designed to produce the prescription.

The prescription is used at 130 to blend a base hair care product with a mixture of botanicals and/or other modifiers. The botanicals are selected and acquired in appropriate amounts according to the prescription directives. Other ingredients can be blended with the customized hair care product that is being produced, such as colorants, pearlescence, and fragrances desired by the individual for whom the customized hair care product is being created, as depicted at 140.

The resulting mixture can be heated (using external or internal chemical agents to a desired or designed temperature) and/or stirred appropriately to achieve a desired consistency and/or texture. At 150, the final resulting mixture represents the customized hair care product. Moreover, at 160, the mixture is filled into a container (e.g., bottle) for delivery to the individual. In one embodiment, at 170, a label is affixed to the container. The label can include the originally generated prescription, as depicted at 180, and/or other verbiage that may be required by law.

One now appreciates how hair care products can be specifically designed, created, and delivered to an individual based on an individual's hair type and sensory desires. This fills a need for many individuals that desire customized hair care treatment and do not like mass produced or impersonal and non customized hair care products.

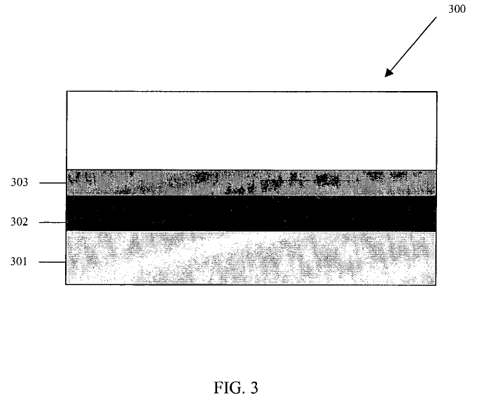

FIG. 3 illustrates a diagram of one customized hair care product 300, according to one embodiment of the present invention. The customized hair care product 300 can be produced entirely in an automated fashion, or in a partially automated fashion. In one embodiment, the customized hair care product 300 is produced by technicians in a salon that are trained to fill prescriptions that create the customized hair care product 300.

The customized hair care product 300 includes at least two components, a base hair care component 301 that represents a concentrated base hair care shampoo, conditioner, and/or combination of shampoo and conditioner. This base hair care component is blended and processed according to normal conventions readily known to one of ordinary skill in the art.

The customized hair care product 300 also includes a second component 302, which represents a mixture of botanicals or other modifiers that are acquired from a prescription. The proper selection, concentration, and mixture of the botanicals 302 are derived from the prescription. The prescription represents the results of an analysis of a specific individual's hair type vis-à-vis a set of factors. The factors can vary depending upon the type of customized hair care product 300 desired by the individual. Moreover, the prescription can identify a percentage of the base hair care product 301 and a percentage of the botanical mixture 302 needed to generate the customized hair care product 300.

In one embodiment, the customized hair care product 300 also includes a number of colorants and/or fragrances 303 that are specified according to the sensory tastes and desires of the individual. These colorants and/or fragrances 303 can be detailed in the prescription, or can be separately identified by the individual. Thus, a prescription can include the proper mixture of botanicals 302 and colorants and/or fragrances 303 can be altered from time to time by the individual without altering the prescription of the medicinal value of the customized hair care product 300.

In some embodiments, the customized hair care product 300 including the base 301, the botanical mixture 302, and optionally any colorants and/or fragrances 303, are heated and stirred together to blend the customized hair care product 300 into a single mixture.

The customized hair care product 300 is specifically designed and formulated based on analysis of a specific individual's hair type. The delivering and packaging of this customized hair care product 300 can also be customized to the needs and tastes of the individual. Moreover, the prescription can be reused by the individual after the initial analysis and resulting prescription are completed. Individuals can now receive specialized and customized products associated with the health needs and sensory desires of the individuals. This is particularly desirable for many individuals that consider hair treatments and products a personal experience and do not desire to purchase products marketed to mass categories of hair needs.

Although specific embodiments have been illustrated and described herein, those of ordinary skill in the art will appreciate that any arrangement calculated to achieve the same purpose can be substituted for the specific embodiments shown. This disclosure is intended to cover any and all adaptations or variations of various embodiments of the invention. It is to be understood that the above description has been made in an illustrative fashion, and not a restrictive one. Combinations of the above embodiments, and other embodiments not specifically described herein will be apparent to one of ordinary skill in the art upon reviewing the above description. The scope of various embodiments of the invention includes any other applications in which the above structures and methods are used. Therefore, the scope of various embodiments of the invention should be determined with reference to the appended claims, along with the full range of equivalents to which such claims are entitled.

It is emphasized that the Abstract is provided to comply with 37 C.F.R. §1.72(b) requiring an Abstract that will allow the reader to quickly ascertain the nature and gist of the technical disclosure. It is submitted with the understanding that it will not be used to interpret or limit the scope or meaning of the claims.

In the foregoing Detailed Description, various features are grouped together in a single embodiment for the purpose of streamlining the disclosure. This method of disclosure is not to be interpreted as reflecting an intention that the claimed embodiments of the invention require more features than are expressly recited in each claim. Rather, as the following claims reflect, inventive subject matter lies in less than all features of a single disclosed embodiment. Thus the following claims are hereby incorporated into the Detailed Description, with each claim standing on its own as a separate preferred embodiment.

1. A method to produce a customized hair care product, comprising:
receiving a base hair care product;
receiving a score for a hair type, wherein the score is associated with a plurality of factors;
using the score to acquire a mixture of botanicals; and
adding the mixture to the base to produce the customized hair product.

2. The method of claim 1 wherein in receiving the score, the plurality of factors include at least 7 factors when the hair care product is a shampoo, and wherein the at least 7 factors include a hair conditioning factor, a hair clarifying factor, a hair medicated factor, a hair body building factor, a relaxant factor, a shine factor, and a color preserving factor.

3. The method of claim 2 wherein in receiving the score, the plurality of factors include at least 4 factors when the hair care product is a conditioner, and wherein the at least 4 factors include a frizz control factor, a color preserving factor, a hair body building factor and a hair deep conditioner factor.

4. The method of claim 1 wherein in adding, the base and the mixture are heated and stirred to produce the customized hair care product.

5. The method of claim 1 further comprising, using other supplied factors to add colors and/or fragrances to the base and the mixture.

6. The method of claim 1 wherein in receiving, the score is received from an analysis of the hair type for which the customized hair product is being customized.

7. The method of claim 1 wherein in receiving, the score is received though interactions with a kiosk.

8. A method to produce a customized hair care product, comprising:
analyzing a hair type based on a plurality of factors;
generating a prescription for a customized hair care product based on scores assigned to the plurality of factors;
blending a base hair care product with a mixture of botanicals determined from the prescription, and
using a resulting mixture from the blending to produce the customized hair care product.

9. The method of claim 8, wherein in analyzing, the hair type is analyzed manually by a hair care specialist.

10. The method of claim 8 wherein in analyzing, the analyzing, the providing, and the blending are performed automatically in a kiosk.

11. The method of claim 8 wherein the blending includes adding colors and/or fragrances to the mixture and the base.

12. The method of claim 8 further comprising, filling a container with the customized hair care product.

13. The system of claim 12 further comprising, affixing a customized label to the container.

14. The method of claim 8 wherein the scores represent numeric values associated with a scale of numbers from 1 to 10, and wherein a single score is assigned to each of the factors, and there are approximately 4 or more factors.

15. A customized hair care product, comprising:
a base hair care product;
a mixture of botanicals determined by a prescription associated with a specific hair type; and
wherein a percentage of the base hair care product and a percentage of the mixture are identified in the prescription in order to produce the customized hair care product.

16. The customized hair care product of claim 15 further comprising an additional mixture of colors and/or fragrances added to the mixture and the base hair care product.

17. The customized hair care product of claim 15, wherein the base hair care product and the mixture are heated and stirred according to instructions provided in the prescription in order to produce the customized hair care product.

18. The customized hair care product of claim 15, wherein the prescription is generated from an analysis of the specific hair type against a plurality of factors, and wherein the factors are altered based on whether the customized hair care product is a shampoo or a conditioner.

19. The customized hair care product of claim 18, wherein the prescription is automatically and electronically generated.

20. The customized hair care product of claim 15, wherein the prescription is received and used to electronically and automatically produce the customized hair care product.
